(19) United States
(12) Patent Application Publication
(10) Pub. No.: US 2014/0322070 A1
(43) Pub. Date: Oct. 30, 2014

(54) HANDHELD DEVICE SANITARY ENCLOSURE SYSTEM AND METHOD FOR USING THE SAME

(71) Applicant: Eric Thomas, Beaverton, OR (US)

(72) Inventor: Eric Thomas, Beaverton, OR (US)

(21) Appl. No.: 13/901,241

(22) Filed: May 23, 2013

Related U.S. Application Data
(60) Provisional application No. 61/815,826, filed on Apr. 25, 2013.

(51) Int. Cl. A61L 2/00

(52) U.S. Cl. CPC A61L 2/00; A61L 2202/182
USPC 422/1; 312/223.4

(57) ABSTRACT

A handheld device sanitary enclosure system includes a system housing including a sanitary enclosure magazine configured to hold a plurality of sanitary enclosures. A support station is coupled with the system housing. The support station is configured to hold and suspend a handheld device at the support station in a hands free manner. The support station suspends the device while the user optionally disinfects or washes their hands. The handheld device sanitary enclosure system further includes an enclosure sealer configured to close a sanitary enclosure of the plurality of sanitary enclosures with a handheld device therein. After enclosure of the handheld device the user may retrieve the enclosed device.

This application claims the benefit of priority under 35 U.S.C. 119(e) to U.S. Provisional Patent Application Ser. No. 61/815,826, filed on Apr. 25, 2013, which is incorporated herein by reference in its entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the data as described below and in the drawings that form a part of this document: Copyright Eric R. Thomas; Beaverton, Oreg., USA; All Rights Reserved.

### TECHNICAL HELD

This document pertains generally, but not by way of limitation, to sanitary storage and handling of handheld devices.

### BACKGROUND

Hygiene practices within any number of industries include hand washing, sterilization of working surfaces and the like. In one example, food service employees are required by restaurant rules or federal or state mandated law to wash hands when beginning or returning to work (e.g., after breaks or restroom use). In other examples, health service employees (care givers including nurses, doctors or the like) are similarly required to consistently wash or disinfect their hands when beginning or returning to work. In still other examples, health service employees are required to wash or disinfect their hands when transitioning between patients.

The use of handheld devices has become common in contemporary society for entertainment as well as work-related purposes. For instance, employees may use one or more applications of a handheld device for emailing, texting or phone calling to conduct job functions. Additionally, handheld devices smart phones, tablet computers or the like) may include one or more work related applications for a variety of services including order tracking, data input, patient monitoring and the like. Accordingly, employees may optionally (in the case of entertainment) or by necessity use a handheld device while performing their jobs.

### Overview

The present inventor has recognized, among other things, that a problem to be solved can include preventing or minimizing the introduction of contaminants, such as bacteria, dirt and the like, to clean environments by way the use of handheld devices including, but not limited to, cell phones, smart phones, tablet computers, data input/output devices and the like. Further the present inventor has recognized that a problem to be solved can include preventing or minimizing the introduction of contaminants to the hands or mouth of a user of a handheld device after the user has previously washed or disinfected their hands. Preventing or minimizing the introduction of contaminants to the hands or mouth of the user correspondingly prevents or minimizes the introduction of the same to surfaces and others the user interacts with (e.g., food, medical devices and equipment, patients and the like).

In an example, the present subject matter can provide a solution to these problems, such as by providing a handheld device sanitary enclosure system (and method for using the same) configured to hold and suspend a handheld device while a user of the device washes or disinfects their hands. The handheld device sanitary enclosure system encloses the handheld device within a sanitary enclosure prior to the user grasping the handheld device. Stated another way, the user delivers a handheld device to the system, the system holds the device therein and encloses the device within a sanitary enclosure. After the user disinfects or washes their hands the handheld device within the sanitary enclosure is retrieved and may thereafter be used without transmitting contaminants to the hands of the user. Equipment, food or patients the user interacts with after use of the enclosed handheld device are similarly protected from any contaminants of the handheld device. Optionally, the handheld device sanitary enclosure system further includes a sterilization feature configured to sanitize the handheld device before, during or after delivery of the handheld device to the sanitary enclosure, for instance by one or more of ultraviolet light sterilization or the application of a disinfectant aerosol or liquid.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a front view of one example of a handheld device sanitary enclosure system.

FIG. 2 is a cross sectional side view of the handheld device sanitary enclosure system of FIG. 1.

FIG. 3A is a front view of another example of a handheld device sanitary enclosure system.

FIG. 3B is a front view of the handheld device sanitary enclosure system of FIG. 3A.

FIG. 4 is a perspective view of yet another example of a handheld device sanitary enclosure system.

FIG. 5A is a cross sectional side view of the handheld device sanitary enclosure system of FIG. 4 with a handheld device at a support station.

FIG. 5B is a cross sectional side view of the handheld device sanitary enclosure system of FIG. 4 including a sterilization feature provided at a delivery port to a sanitary enclosure.

FIG. 5C is a cross sectional side view of the handheld device sanitary enclosure system of FIG. 4 including a shrink wrapping enclosure sealer.

FIG. 6 is a block diagram showing one example of a method for using a handheld device sanitary enclosure system.

### DETAILED DESCRIPTION

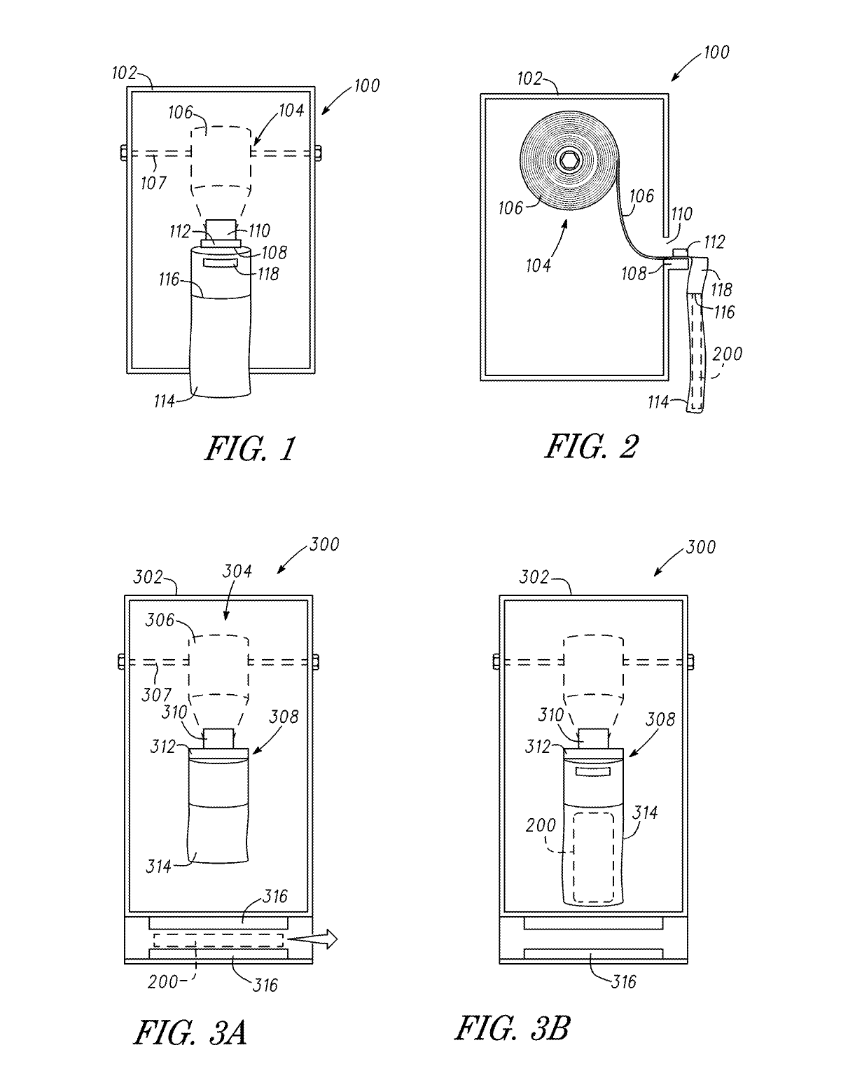

FIG. 1 shows one example of a sanitary enclosure system 100. In the example, the sanitary enclosure system 100 includes a system housing 102 including a sanitary enclosure magazine 104 therein. The sanitary enclosure magazine 104 includes but is not limited to a plurality of sanitary enclosures 106 retained within the system housing 102. For instance, in one example the plurality of sanitary enclosures 106 are provided in a roll form, such as, tubular roll of interconnected bags, tubes or the like. Optionally, the tube includes, but is not limited to including, perforations or the like to facilitate separation of portions of the roll (e.g., as the sanitary enclosures 106). As shown in the example of FIG. 1, the plurality of sanitary enclosures 106 are provided in a rolled format and provided within the sanitary enclosure magazine 104 on a spool 107. The spool 107 allows for retaining of the enclosures and dispensing of the enclosures within the system housing 102. In another example, the plurality of sanitary enclosures 106 are retained as a stack within a box-like structure within the system housing 102, such as a shelf or other feature.

The plurality of sanitary enclosures 106 are connected in one example by perforated joints (e.g., the plurality of sanitary enclosures are provided in a continuous tube with perforations provided along the tube). In another example the plurality of sanitary enclosures 106 are folded with one another (e.g., in a manner similar to folded paper towels). Accordingly, upon drawing of a preceding sanitary enclosure from the sanitary enclosure magazine 104 a proceeding or following sanitary enclosure 106 is fed from a stack of the plurality of sanitary enclosures and thereafter delivered through the system housing 102 (e.g., to the enclosure dispensing orifice 110 described herein below).

As further shown in FIG. 1 and also shown in FIG. 2, the plurality of sanitary enclosures 106 are fed through an enclosure dispensing orifice 110 through an exterior of the system housing 102. As shown in FIG. 2, in one example the plurality of sanitary enclosures 106, such as a single sanitary enclosure 114, extends through the enclosure dispensing orifice 110 and extends from the system housing 102 in a substantially vertical manner. In one example, the enclosure dispensing orifice 110 has a smaller diameter, width or the like relative to a width of one of the sanitary enclosures of the plurality of sanitary enclosures 106. Accordingly, the enclosure dispensing orifice 110 throttles the passage of the enclosures (e.g., provides friction) and accordingly allows for the retention of the sanitary enclosures 106 as they are drawn through the enclosure dispensing orifice 110. Additionally, the resistance provided at the enclosure dispensing orifice 110 optionally maintains tension on the plurality of sanitary enclosures 106 within the magazine 104 and accordingly prevents or minimizes the development of stack. Upon pulling a single sanitary enclosure from the sanitary enclosure magazine 104 the enclosure dispensing orifice 110 (and optionally the enclosure hanger 112 described herein) ensure that the remainder of the plurality of sanitary enclosures 106 remain stored within the sanitary enclosure magazine 104 until pulled through the enclosure dispensing orifice 110.

Referring again to FIG. 1, one of the sanitary enclosures of the plurality of sanitary enclosures is shown in a substantially vertical orientation relative to the system housing 102. As shown, the sanitary enclosure 114 extends downwardly with an enclosure opening 116 (e.g., a mouth) positioned near to a support station 108. The enclosure opening 116 includes, in one example, a slit or cut formed along one surface of the tubular roll of the plurality of sanitary enclosures 106. In one example the support station 108 includes but is not limited to an enclosure hanger 112, such as an enclosure clamp, sized and shaped to provide a frictional engagement with the plurality of sanitary enclosures 106. Accordingly, the sanitary enclosure 114 of the plurality of sanitary enclosures 106 is suspended from the support station 108 even with a handheld device positioned within the enclosure.

As shown in FIG. 1, the sanitary enclosure 114 is suspended below the support station 108 with the enclosure opening 116 in a revealed position. A user may readily deposit a handheld device within the sanitary enclosure 114 before the user disinfects or washes their hands and prior to sealing of the handheld device within the enclosure 114. As previously described herein, in one example the enclosure hanger 112 at the support station 108 includes an enclosure clamp, for instance a clamp providing a frictional engagement with the sanitary enclosure 114. The enclosure clamp permits movement of the sanitary enclosure 114 through the enclosure hanger 112, for instance with tension applied by putting of the user. At the same time, once tension is interrupted to the sanitary enclosure 114 the sanitary enclosure 114 (including a handheld device 200 therein) is suspended from the support station 108 by the enclosure hanger 112. In another example the enclosure hanger 112 includes but is not limited to a mechanical clip, anchor, pin or the like.

As further shown in FIGS. 1 and 2 the sanitary enclosure system 100 includes in another example an enclosure sealer 118. In the example shown in FIGS. 1 and 2 the enclosure sealer 118 includes, but is not limited to, an adhesive strip applied to each of the sanitary enclosures 114 of the plurality of sanitary enclosures 106 provided in the sanitary enclosure magazine 104. As shown for instance in FIG. 1 the adhesive strip is applied to a portion of the sanitary enclosure 114, such as a flap, extending above that portion of the sanitary enclosure 114 providing a pocket for reception of a handheld device therein. Accordingly, upon removal of the sanitary enclosure 114 from the system housing 102 (e.g., after washing or disinfecting of the hands of the user) the adhesive strip 118 is applied to the remainder of the sanitary enclosure 114 for instance by folding of a flap of the sanitary enclosure over top of the remainder of the sanitary enclosure to seal the handheld device therein.

In another example the sanitary enclosure system 100 includes an enclosure sealer 118 providing a differing seal to the sanitary enclosure 114. In one example and as described further herein, the enclosure sealer 118 includes a heat sealer sized and shaped to engage the sanitary enclosure 114 and thereby provide a heat seal at the enclosure opening 116 to accordingly seal the handheld device within the sanitary enclosure 114. In another example, the heat sealer includes a heating element (or elements) configured to heat the enclosure 114 constructed with a heat shrinking material to shrink wrap and seal the device. Optionally, the heat sealer is included with the support station 108, such as the enclosure hanger 112, and is selectively operated according to a timer or user interaction (e.g., passage of the hand over a photo eye, infrared sensor or the like). In another example the enclosure sealer 118 is included for instance with the plurality of sanitary enclosures 106. For instance each of the sanitary enclosures 114 is constructed with but not limited to a material having an adhesive property for instance a material similar to an adhesive plastic. The plastic of the sanitary enclosure 114 accordingly engages and provides a fixable coupling with itself to close the enclosure opening 116, for instance by folding of a flap of the sanitary enclosure 114 over top of the remainder of the enclosure.

Referring now to FIG. 2, in operation a handheld device 200 is deposited within the sanitary enclosure 114 extending from the system housing 102. That is to say, the handheld device 200 is positioned within the sanitary enclosure 114 by positioning of the handheld device through an enclosure opening 116. After positioning of the handheld device 200 within the sanitary enclosure 114 the user optionally disinfects or washes their hands. After sufficient cleaning of the hands the user removes the handheld device 200 and the surrounding sanitary enclosure 114 from the system housing 102. For instance, the enclosure hanger 112 such as an enclosure clamp provides frictional engagement with the remainder of the plurality of sanitary enclosures 106 to allow ready removal of the sanitary enclosure 114 from the remainder of the plurality of sanitary enclosures 106. Optionally, plurality of sanitary enclosures 106 are interconnected with a series of perforations and tension provided by the user to the sanitary enclosure 114 accordingly separates the sanitary enclosure 114 from the plurality 106 by fracture of the perforations. The user then seals the sanitary enclosure 114 for instance with the enclosure sealer 118, such as an adhesive strip. In one example, a flap of the sanitary enclosure 114 is folded over the remainder of the sanitary enclosure and an adhesive strip 118 is used to seal the sanitary enclosure and thereby isolate the handheld device 200 within the sanitary enclosure 114 from further interaction by the user. While received and enclosed within the sanitary enclosure 114, the sanitary enclosure 114 allows the user to interact with the handheld device 200 through a film material (e.g., polymer bags, FDA approved sterile polymer bags, medical grade plastic, shrink wrap films or bags, vacuum sealing bags or the like) of the enclosure 114. The film material of the enclosure 114 otherwise provides a sanitary sleeve to the handheld device that substantially prevents the transmission of contaminants between the handheld device 200 and the hands or mouth of the user and other clean or sanitary surfaces within a clean environment.

In another example the enclosure hanger 112 provides a separation feature configured to separate the sanitary enclosure 114 from the plurality of sanitary enclosures 106. For instance, in one example the enclosure hanger 118 provides a serrated or cutting edge sized and shaped to separate the sanitary enclosure 114 from the remainder of the plurality of sanitary enclosures 106 upon the application of tension by pulling on the sanitary enclosure 114. In still another example (and as described above), the enclosure hanger includes a heat sealing enclosure sealer. Operation of the heat sealing enclosure sealer (e.g., the heating of one or more heated elements engaged with the enclosure) seals the enclosure 114 and at the same time separates it from the plurality of the sanitary enclosures 106.

FIGS. 3A and 3B show another example of a sanitary enclosure system 300. The sanitary enclosure system 300 includes similar features to the previously described sanitary enclosure system 100 (FIGS. 1 and 2). For instance, the sanitary enclosure system 300 includes a system housing 302 as well as a sanitary enclosure magazine 304 sized and shaped to retain a plurality of sanitary enclosures 306 therein. In one example, the sanitary enclosure magazine 304 includes a roll of sanitary enclosures for instance a roll of bag-like sanitary enclosures or a continuous tube of sanitary enclosures separated by separation features, such as perforations or the like rolled around a spool 307 within the system housing 302. As further shown in FIG. 3A, the plurality of sanitary enclosures 306 are fed through an enclosure dispensing orifice 310. A sanitary enclosure 314 in a partially presented position is shown in FIG. 3A. The sanitary enclosure 314 is retained at a support station 308, for instance by an enclosure hanger 312.

In one example, the sanitary enclosure system 300 includes an enclosure sealer for closing of an enclosure after a handheld device 200 is provided therein. For instance, as shown in FIGS. 3A, B, the sanitary enclosures 314 include an enclosure sealer 118, such as an adhesive strip or the like, configured to close the sanitary enclosure 314 upon removal from the remainder of the plurality of sanitary enclosures 306 (e.g., by separation at the enclosure hanger 312 such as by separation of perforations, cutting or the like (e.g., heating of the sanitary enclosure material) at the support station 308). As previously described above in the example of the enclosure hanger 112 shown in FIGS. 1 and 2, optionally the enclosure hanger 112 includes a heat sealer that closes the enclosure 114 while at the same separating the enclosure from the plurality of the sanitary enclosures. The enclosure hanger 312, in another example, includes a similar enclosure sealer. In still another example, the enclosure sealer includes a heating element (or elements) provided along the system housing 302 and configured to heat the enclosure constructed with a heat shrinking material to shrink wrap and seal the device.

As further shown in FIG. 3A, the sanitary enclosure system 300 includes a sterilization feature 316. The handheld device 200 is positioned within the sterilization feature 316. The sterilization feature 316 provides one or more mechanisms configured to sterilize the handheld device 200 prior to positioning within the sanitary enclosure 314. In the example shown in FIG. 3A the sterilization feature 316 includes one or more of ultraviolet (UV) light generating lamps configured to bathe the handheld device 200 in ultraviolet light for a period of time and thereby sterilize the handheld device 200 prior to positioning within the sanitary enclosure 314. Optionally, the sterilization feature 316 includes alternative or supplemental sterilization features including, but not limited to, an aerosol sanitizing dispenser, a liquid sanitizing dispenser, a wipe or brush applicator for applying a sanitizer or the like. In still another example, the sterilization feature 316 provides a supporting feature (e.g., a shelf, chamber or cavity) configured to hold the handheld device 200 while the user sanitizes or washes their hands. Accordingly, the sterilization feature is optionally used as the support station (alternatively or in addition to the support station 308) for the device while the user is washing or disinfecting their hands.

In operation, the user positions the handheld device 200 within the sterilization feature 316. For instance, the sterilization feature 316 includes a cavity, shelf, chamber or the like provided within the system housing 302 sized and shaped to receive the handheld device 200 therein. While the handheld device 200 is retained within the sterilization feature 316 the user disinfects or washes their hands. In one example the sterilization feature 316 includes but is not limited to an ultraviolet light source, a disinfectant source such as an aerosol spray, gel or the like including an applicator (e.g., a brush or wipe) configured to apply the disinfectant to the handheld device 200 while within the sterilization feature 316. In one example, the sterilization feature 316 is included with or is by itself part of the support station configured to suspend the handheld device 200 prior to interaction again by the user. For instance the sterilization feature 316 provides a feature for storage of the handheld device 200 while the user disinfects or cleans their hands.

Referring now to FIG. 3B after disinfecting of the handheld device 200 for instance with the sterilization feature 316 the handheld device 200 is positioned within the sanitary enclosure 314 hanging from the support station 308 by way of an enclosure hanger 312. In the example shown in FIG. 3B, the operation of the sanitary enclosure system 300 at this stage is similar in at least some regards to the operation of the sanitary enclosure system 100 previously shown and described in FIGS. 1 and 2. That is to say, the now sanitized handheld device 200 is positioned within the sanitary enclosure 314 and upon tensioning of the sanitary enclosure 314 and interaction for instance with a cutting surface, heating surface, perforations or the like the enclosure is removed from the remainder of the plurality of sanitary enclosures 306. The enclosure sealer, for instance an adhesive strip is used to close the sanitary enclosure 314 and thereby seal the handheld device 200 therein. Optionally, where the enclosure sealer includes a heat sealer (e.g., incorporated within the enclosure hanger 312) the sealer seals the enclosure 314 white at the same time separating the enclosure from the plurality of sanitary enclosures (and sealing the bottom of the proceeding enclosure).

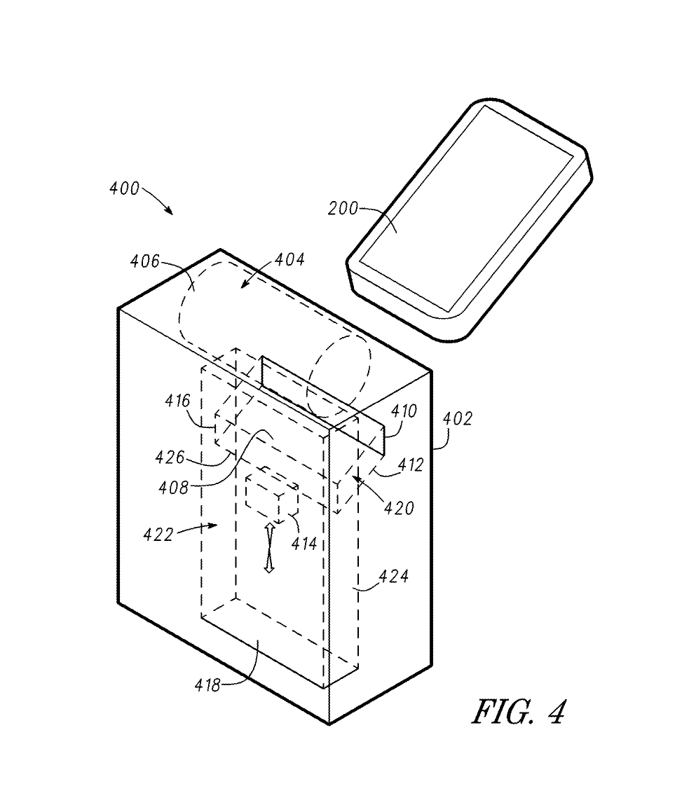

Referring now to FIG. 4, another example of a sanitary enclosure system 400 is provided. In at least some regards the sanitary enclosure system 400 is similar to the previously described enclosure systems 100, 300 provided herein. For instance, the sanitary enclosure system 400 includes a system housing 402 and a sanitary enclosure magazine 404. In contrast to the previously described sanitary enclosure systems, the suspending, sanitizing and enclosing features of the sanitary enclosure system 400 are contained within the system housing 402 thereby allowing a user to deposit a phone within the sanitary enclosure system 400 and the sanitary enclosure system 400 proceeds to sanitize and then enclose the handheld device such as the handheld device 200 within a sanitary enclosure while the user is disinfecting or washing their hands.

The sanitary enclosure system 400 shown in FIG. 4 includes an inlet port 410 provided on at least one surface of the system housing 402. As shown, the inlet port 410 opens into a support station 408. In the example shown, the support station 408 includes a conveying surface 412. In one example, the conveying surface 412 has a slope and a conveying stop 414 is provided at one end of the conveying surface 412 (e.g., adjacent to a delivery port 416). As will be described herein, the conveying stop 414 is moveable into an intercepting configuration where the conveying stop 414 is positioned at least in part within the delivery port 416 or above a portion of the conveying surface 412 to intercept a handheld device 200 positioned through the inlet port 410 and supported upon the conveying surface 412. The conveying stop 414 arrests movement of the handheld device and accordingly suspends the handheld device on the conveying surface 412 (e.g., for sanitizing).

As further shown in FIG. 4, the sanitary enclosure system 400 includes a sanitizing zone 420 and an enclosing zone 422. In one example, the sanitizing zone 420 is included with the support station 408. For instance, the sanitizing zone 420 includes one or more sterilization features configured to sanitize a handheld device 200 positioned within the inlet port 410 and held on the conveying surface 412. In contrast, the enclosing zone 422 provides one or more mechanisms for receiving and enclosure a handheld device 200 within one or more sanitary enclosures for instance a sanitary enclosure of a plurality of sanitary enclosures 406 for instance retained within a sanitary enclosure magazine 404 as also shown in FIG. 4. The handheld device is accordingly retained and conveyed between each of the sanitizing and enclosing zones 422 (while in each of the zones the handheld device is accordingly retained in respective sanitizing and enclosing configurations).

In one example the enclosing zone 422 includes an enclosure chute 424 extending between an outlet port 418 and the sanitary enclosure magazine 404. The enclosures of the plurality of sanitary enclosures 406 are fed through the enclosure chute 424 for instance by an automated mechanism, such as a motor coupled with a spool of the plurality of sanitary enclosures 406, or the plurality of sanitary enclosures are pulled by the user as the user removes an enclosed handheld device through the outlet port 418. As will be described herein, the plurality of sanitary enclosures 406 serially deliver sanitary enclosures for receipt of handheld devices delivered from the delivery port 416 for instance into enclosure openings (mouths) of each of the sanitary enclosures.

In another example and as wilt be described in further detail herein, the outlet port 418 includes an enclosure sealer, for instance a heat sealer configured to heat seal the sanitary enclosure containing a handheld device therein. 111 yet another example, the heat sealer includes a heating element (or elements) configured to heat the enclosure (constructed with a heat shrinking material) to shrink wrap and seal the device. In still another example, the outlet port 418 includes a retention clip, such as a frictional retention clip, configured to hold the phone within the sanitary enclosure therein until interaction by the user (e.g., pulling of the sanitary enclosure and cutting or separation of perforations within the sanitary enclosure from the remainder of the plurality of sanitary enclosures 406).

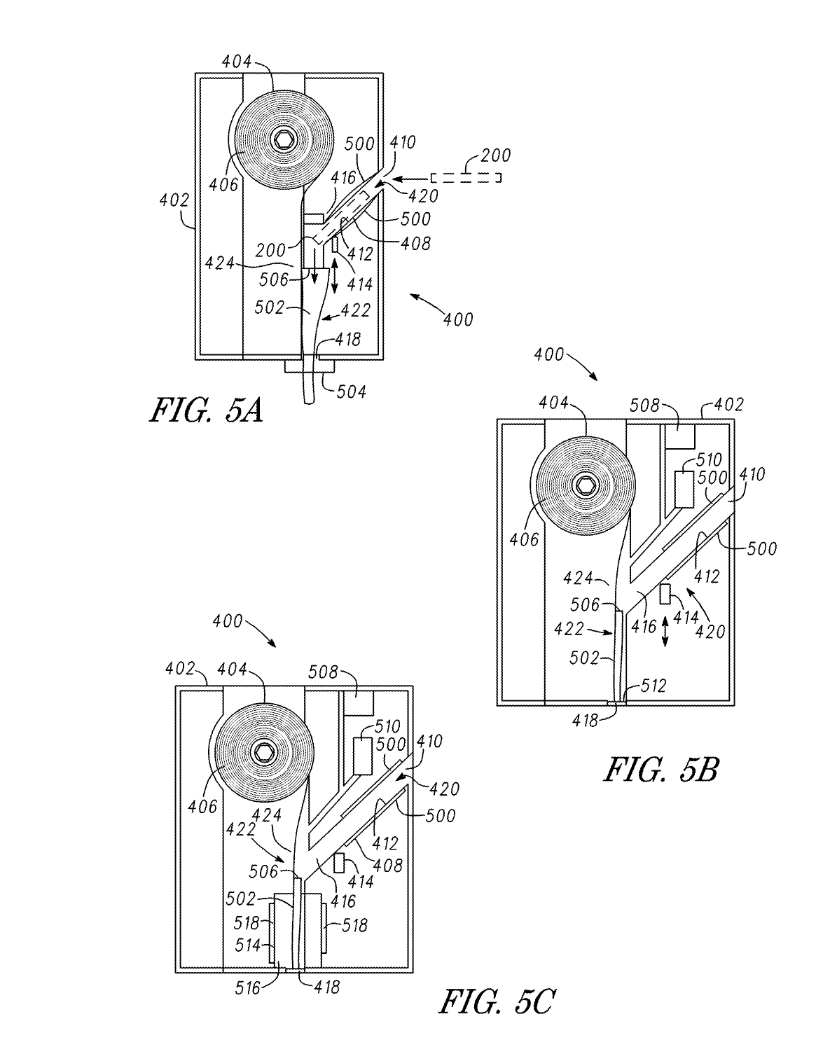

Referring now to FIG. 5A the sanitary enclosure system 400 is shown in a first configuration including an enclosure hanger 504 positioned at the outlet port 418. As further shown in FIG. 5A, a sterilization feature 500 such as one or more ultraviolet lamps is provided within the sanitizing zone 420, for instance adjacent to the conveying surface 412. Dual sterilization features 500 (e.g., dual ultraviolet lamps) are provided on either side of a handheld device 200 positioned on the conveying surface 412 when the device 200 is delivered through the inlet port 410. Optionally, the sterilization features 500 include, but are not limited to, sanitizing applications such as a wipes or brushes, liquid or aerosol dispensers or the like.

The conveying stop 414 is also shown in a depressed configuration in FIG. 5A thereby allowing the handheld device 200 (the second instance of the device 200 in broken lines) to slide along the conveying surface 412 into the enclosure chute 424 for receipt within a sanitary enclosure, such as the enclosure 502. In this configuration the sanitary enclosure system 400 includes an enclosure hanger 504 positioned at the outlet port 418. As shown, the enclosure hanger 504 engages the handheld device 200, for instance with a frictional engagement along the enclosure 502. The enclosure hanger 504 further provides tension to the plurality of sanitary enclosures 406 and accordingly maintains at least a single proceeding enclosure 502 (following the preceding enclosure removed through the outlet port 418) at a position wherein the handheld device 200 delivered from the conveying surface 412 is readily received through the enclosure mouth 506 into the enclosure 502. In another example the system housing 402 is sized and shaped according to the dimensions of the enclosures 502 to accordingly position the enclosure mouth 506 adjacent to the delivery port 416. Accordingly as handheld devices 200 are fed through the sanitary enclosure system 400, sanitized and then enclosed within preceding enclosures and removed from the sanitary enclosure system 400 the proceeding enclosure 502 is moved into a ready position for instance with the enclosure mouth 506 adjacent to the delivery port 416 and ready to receive another handheld device 200.

Referring again to FIG. 5A, the sanitary enclosure system 400 including is described in operation. A handheld device 200 is delivered through the inlet port 410 into the system housing 402. In the example shown in FIG. 5A the handheld device 200 slides along the conveying surface 412 for instance into engagement with the conveying stop 414 (provided in an upward orientation relative to that shown in FIGS. 4 and 5A). In one example the conveying stop 414 is actuated by a stepper motor, solenoid or the like to provide an intercepting feature for the handheld device 200 to arrest its movement along the conveying surface 412 and accordingly hold the handheld device 200 at a support station 408 for instance formed by the conveying surface 412. In one example the conveying stop 414 holds the handheld device 200 along the conveying surface 412 while the sterilization features 500 sanitize the handheld device 200 for a set amount of time. Stated another way, the system 400 is in a sanitizing configuration with the hand held device 200 suspended at the support station 408. In one example the sterilization features 500 are configured to bathe the handheld device 500 in ultraviolet light for 5, 10, 15 or 20 seconds or longer to accordingly sterilize the handheld device 200 prior to enclosure in the enclosing zone 422. Optionally, other sterilization features including, but not limited to, brushes, wipes, aerosol or liquid dispensers sterilize the handheld device 200 for sufficient time to sterilize the device.

After sanitizing is completed (e.g., the desired amount of time has passed), in one example an algorithm within the sanitary enclosure system 400 for instance a controller therein depresses the conveying stop 414 and accordingly allows the handheld device 200 to move through the delivery port 416 into the enclosure chute 424. As shown in FIG. 5A, the system housing 402, the enclosure chute 424 and the delivery port 416 position along the chute 424 are dimensioned to ensure the enclosure mouth 506 of the enclosure 502 is positioned adjacent to the delivery port 416. Accordingly, in an enclosing configuration of the system 400 the handheld device 200 is automatically delivered through the delivery port 416 through the enclosure mouth 406 and into the enclosure 502.

As further shown in FIG. 5A, with the handheld device 200 received within the enclosure 502 the enclosure hanger 504 (for instance at the outlet port 418) engages with the enclosure 502 and accordingly holds the enclosure in place. The user (after disinfecting or washing their hands) reaches for the handheld device and pulls it from the system housing 402. In one example the friction provided by the enclosure hanger 504 engages with the proceeding enclosure 502 of the plurality of sanitary enclosures 406 and provides sufficient friction to the plurality of sanitary enclosures 406 to accordingly allow for tearing of the enclosure 502 including the handheld device 200 therein from the remainder of the plurality of sanitary enclosures 406.

Upon removal of the enclosure 502 with the handheld device 200 therein the handheld device 200 is sanitized and positioned within an enclosure 502. The user then optionally seals the enclosure 502 for instance by applying an adhesive strip over top of the remainder of the enclosure 502 to close the enclosure 502 and seal the handheld device 200 (in a sanitized configuration) therein.

FIG. 59 shows a cross-sectional view of the sanitary enclosure system 400 in a second configuration. Many of the features of the second configuration of the sanitary enclosure system 400 are similar to the previously described features. For instance, the sanitary enclosure system 400 of FIG. 59 includes a sanitizing zone 420 and enclosing zone 422. In addition, the sanitizing zone 420 receives a handheld device 200, for instance through the inlet port 410. The sanitizing zone includes a conveying surface 412 in communication with a delivery port 416 and the delivery port 416 opens into the enclosure chute 424 including an enclosure 502 with an open enclosure mouth 506 therein.

In the example shown in FIG. 59, a supplemental or alternative sterilizing feature is provided. As shown, the sterilization feature includes a sanitizer source 510 and a pressurized fluid source 508. In one example the pressurized fluid source includes, but is not limited to, a fan, source of compressed air such as a compressed gas tank, compressor or the like. The sanitizer source 510 is positioned adjacent to an outlet of the pressurized fluid source 508 (e.g., downstream from the outlet). Accordingly, as an enclosure 502 is fed from the sanitary enclosure magazine 404, for instance by an automatic or mechanical feature coupled with the sanitary enclosure magazine 404, the sanitizer source 510 cooperates with the pressurized fluid source 508 to sanitize the interior of the enclosure 502 and thereby provide a sanitary environment for reception of a handheld device 200 therein. For instance, in one example the sanitizer source 510 and the pressurized fluid source 508 act in cooperation with the mechanism operating the sanitary enclosure magazine 404. As the sanitary enclosure magazine 404 feeds out one or more sanitary enclosures 502 the sanitizer source and the pressurized fluid source 510, 508 automatically act to provide a puff or injection of pressurized fluid into the enclosure 502 for instance through the enclosure mouth 506 along with a sanitizing fluid for instance provided by the sanitizer source 510. That is to say, the pressurized fluid source 508 entrains the sanitizer from the sanitizer source 510 and provides the entrained fluid to the enclosure 502 for instance the interior therein. Optionally, the injection of pressurized fluid reliably opens the enclosure mouth 506 to ensure the enclosure 502 is open and ready to receive a handheld device 200 form the delivery port 416.

In another example, the sterilization feature previously described (e.g., the sanitizer source 510) acts in cooperation with another sterilization feature, such as the sterilization feature 500 previously shown in FIG. 5A. That is to say, in one example the conveying surface 412 of the sanitary enclosure system 400 shown in FIG. 5B includes the conveying stop 414. Accordingly, a handheld device 200 provided through the inlet port 410 is arrested along the conveying surface 412 to accordingly sanitize the device therein. After sanitizing of the handheld device 200, the conveying stop 414 is depressed (e.g., by an automated controller with a timing function) to allow the device 200 to move through the delivery port 416, for instance into the enclosure mouth 506 of the enclosure 502.

In another example (also shown in FIG. 5B) the sanitary enclosure system 400 includes an enclosure sealer provided at the outlet port 418. The enclosure sealer includes a heat sealer 512 provided at the outlet port 418. In one example, the sanitary enclosure system 400 includes a controller in communication with a proximity switch, photo eye, infrared sensor or the like (e.g., a sensor) configured to detect the presence (or passage) of a sanitary enclosure 502 and a handheld device 200 therein. As the user interacts with the handheld device 200, for instance to pull it from the remainder of the system housing 402 the sensor detects an end of the handheld device 200 and the heat sealer accordingly operates to seal the enclosure 502, for instance by a pinching operation wherein one or more heated elements engage against and heat seal the sanitary enclosure 502. Optionally, the sanitary enclosure magazine 404 lowers the enclosure 502 and the device 200, and the sensor detects the passage of the device 200 and accordingly operates the heat sealer 512.

In still another example, the enclosure sealer 512 (e.g., a heat sealer) operates according to interaction by the user. For instance, as the user moves his hand underneath the system housing 402 a photosensitive eye, infrared sensor or other feature configured to detect the hands notes the presence of the hands and operates the sanitary enclosure magazine 404 to lower the enclosure 502 with the handheld device 200 therein. The enclosure sealer 512 operates after dispensing of the handheld device 200 (e.g., through another sensor as described herein) to seal the end of the enclosure 502 for instance by engaging a heated element against the enclosure 502 to seal the enclosure 502.

In operation, the sanitary enclosure system 400 as shown in FIG. 5B operates in at least some regards similarly to the previously described embodiments herein. For instance, the handheld device 200 is provided through the inlet port 410 and conveyed along the conveying surface 412 for instance to a conveying stop 414 configured to intercept the handheld device 200. The optional sterilization features 500, for instance ultraviolet lamps, bathe the handheld device 200 in ultraviolet light for a period of time configured to disinfect the handheld device 200. At the same time, the sanitizer source 510 (e.g., the second or alternative sterilization feature) cooperates with the pressurized fluid source 508 to provide an entrained flow of sanitizer in a fluid, such as air, pressurized gas or the like to the enclosure 502 (or optionally the handheld device 200 as it enters the enclosure). The entrained sanitizer is received within the enclosure 502 for instance through the enclosure mouth 506. Optionally, the flow of pressurized fluid including the entrained sanitizer reliably opens the enclosure 502 and accordingly maintains the enclosure mouth 506 in an open configuration to receive the handheld device 200. In another example, the enclosure mouth 506 remains open according to tension applied to the enclosure 502 between the outlet port 418 and the sanitary enclosure magazine 404. That is to say, the outlet port 418 grasps the enclosure 502 and holds it taut in combination with the sanitary enclosure magazine 404.

After sterilization of the handheld device 200 within the sanitizing zone 420 (e.g., the sanitizing configuration of the system 400) the conveying stop 414 is depressed to allow the handheld device 200 to move through the delivery port 416 and into the enclosure 402 (e.g., into the enclosure configuration of the system 400). As previously described, a sanitary clean environment is provided within the enclosure 502 for instance by the sanitizer source 510 entrained within the pressurized fluid. In one example, upon receipt of the handheld device 200 within the enclosure 502 the sanitary enclosure magazine 404 automatically operates to lower the handheld device 200 and the enclosure 502 to accordingly allow for removal and optional sealing of the enclosure 502. For instance, a proximity switch, optical sensor, photo eye or the like is provided within the system housing 402. Upon lowering of the handheld device 200 past one or more of the sensors the enclosure sealer 512 (e.g., a heat sealer) is configured to engage with the sanitary enclosure 502 and close the sanitary enclosure 502 to seal the handheld device 200 therein. In another example, the interaction of the user for instance the positioning of a hand adjacent to the outlet port 418 or underneath the system housing 402 is detected by a photosensitive eye, sensor or other feature that accordingly operates the sanitary enclosure magazine 404 to lower the enclosure 502 with the handheld device 200 therein and operates the enclosure sealer 512 to seal the enclosure 502 around the handheld device 200. In still another example, the user pulls the enclosure 502 and the device 200 partially extending through the outlet port 418. A sensor within the enclosure chute 424 detects the passage of the device 200 and operates the enclosure sealer 512 to seal the enclosure 502 around the device 200.

As described above, the enclosure sealer 512 includes a heat sealer in one example configured to engage and accordingly seal the enclosure 502. In another example, the plurality of sanitary enclosures 406 are constructed with a heat shrinking material. In this example, the enclosure sealer 512 includes one or more heating elements configured to heat the enclosure 502 and thereby shrink wrap the enclosure around the device 200.

In still another example, the sanitary enclosure system 400 includes a shrink wrap sealer 514 as another option for an enclosure sealer. The sanitary enclosure system 400 shown in FIG. 5C includes at least some of the features of the previously described sanitary enclosure systems. For instance, the system 400 shown in FIG. 5C includes a sanitizing zone 420 and an enclosing zone 422. As shown in FIG. 5C, the sanitizing zone includes a support station 408 including a conveying surface 412 (e.g., a sliding surface) and optionally includes one or more sterilization features 500, such as UV lamps. A conveying stop 414 is movable relative to the conveying surface 412. In a sanitizing configuration (e.g., where a handheld device is received on the conveying surface 412) the conveying stop 414 intercepts the handheld device and holds it in place during sanitizing, for instance with the sterilization features 500. Withdrawal of the conveying stop 414 allows the handheld device to move along the conveying surface 412, through the delivery port 416 and into the enclosing zone 422.

In the enclosing zone 422 optional sterilization features are provided to sterilize the enclosure 502 (including the interior of the enclosure). In one example, the sterilization features include, but are not limited to the pressurized fluid source 508 in combination with the sanitizer source 510. As previously described, the pressurized fluid source includes a fan, blower, pressurized fluid tank or the like configured to provide a flow of fluid past the sanitizer source 510. The flow of fluid entrains the sanitizer and delivers the entrained sanitizer to the enclosure 502, for instance, the enclosure mouth 506. The velocity of fluid opens the enclosure mouth 506 and holds the enclosure mouth open and ready to receive the handheld device provided through the delivery port 416.

As previously described, the sanitary enclosure system 400 shown in FIG. 5C includes an enclosure sealer, such as the shrink wrap sealer 514. The shrink wrap sealer 514 includes a passage within the system house 402 extending toward the outlet port 418. In one example, the passage is a heat tunnel 516 configured to supply heat to the enclosure 502 after the enclosure receives the handheld device. The heat tunnel 5116 includes one or more heat sources, such as heating elements 518. The heating elements 518 include, but are not limited to, radiative heating elements, convective heat sources or the like. The heating elements provide one or more of radiative heat, a flow of heated fluid such as air, or the like to the enclosure 502 to heat and accordingly shrink the enclosure 502 around the handheld device. The handheld device is closed (e.g., sealed) within the enclosure 502 (and the enclosure is optionally intimately engaged along the surfaces of the handheld device). In the example of radiative heating elements, the heating elements 518 include one or more of resistive heating elements, infrared lamps or the like. In the example of convective heating elements, the heating elements 518 include, but are not limited to, resistive heating elements and a source of fluid, such as a blower, that forces a stream of fluid over the heating elements and directs the heated stream over the enclosure 502.

The enclosure 502 as well as the plurality of sanitary enclosures 406 (e.g., a tubular roll of the enclosures 502) include heat shrinkable materials constructed with, but not limited to, polyolefin, PVC, laminates of polymers or the like. Optionally, the heat shrinkable material is crosslinked, or non-crosslinked. Coextrusions and lamination of the materials are conducted for barrier properties (e.g., to shrink wrap for clean environments, kitchens, medical environments and the like). For example, a multi-layer heat shrinkable laminate includes EP/EVA/copolyester/EVA/EP, where EP is ethylene-propylene and EVA is ethylene-vinyl acetate copolymer.

In operation, the handheld device 200 is provided through the delivery port 416 and received within the enclosure 502 through the enclosure mouth 506. The enclosure 502 is received within the heat tunnel 516 of the shrink wrap sealer 514. Optionally, the shrink wrap sealer 514 includes a sensor or other feature (e.g., a photo eye, infrared sensor, proximity switch or the like) that detects the presence of the handheld device 200 and initiates heating within the heat tunnel 516. The heating elements 518 heat the enclosure 502 with the handheld device 200 therein and close the enclosure around the handheld device. The heating elements 518 heat the enclosure 502 to a temperature sufficient to shrink wrap while at the same time preventing damage to the handheld device. In one example, the shrink wrap sealer 514 operates for a specified amount of time (e.g., 5, 10, 15 or 20 seconds) to shrink the enclosure 502 around the handheld device 200.

Optionally, the user grasps the closed handheld device 200 extending through the outlet port 418 and pulls handheld device through the port after heating. Pulling of the closed handheld device 200 moves the next enclosure 502 into a ready position relative to the delivery port 416 (e.g., pulls the next enclosure from the magazine 404 such as a spool or stack of enclosures). In another option, the sanitary enclosure magazine 404 (e.g., a motorized spool holding the plurality of sanitary enclosures 406) is operated to lower the enclosure 502 and the handheld device 200 therein through the outlet port 418 after heating. In one example, at the termination of heating a controller, switch or other feature triggers the operation of the sanitary enclosure magazine 404 to lower the enclosed handheld device 200 through the outlet port 418 (and also position the next enclosure in a ready position to receive another handheld device).

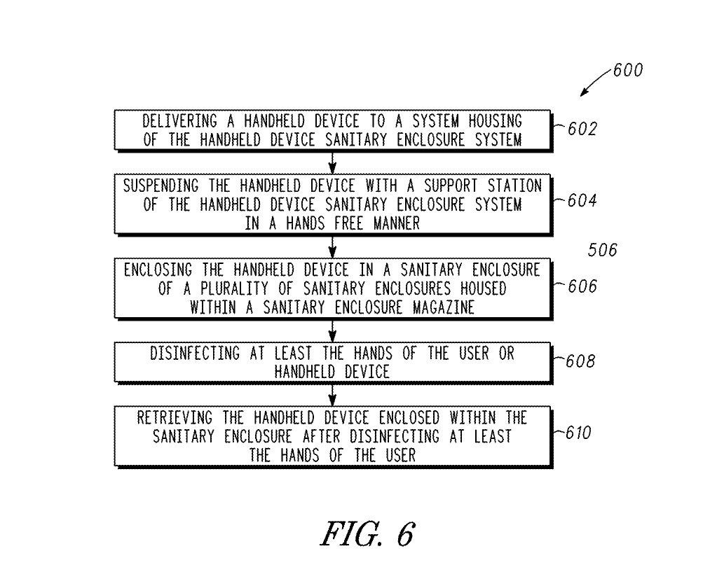

FIG. 6 shows a block diagram for a method 600 for using a handheld device sanitary enclosure system, such as the systems described herein. In describing the method 600 reference is made to one or more components, features, functions, steps or the like described herein. Where convenient, reference is made to the components, features, functions, steps or the like with reference numerals. The reference numerals provided are exemplary and are not exclusive. For instance, the components, features, functions, steps or the like described in the method 600 include, but are not limited to, the numbered elements corresponding to the elements described herein (both numbered and unnumbered) as well as their equivalents.

At 602, the method 600 includes delivering a handheld device 200 to a system housing 102 of the handheld device sanitary enclosure system 100. In one example, delivering the handheld device 200 to the system housing includes delivering the handheld device 200 through an inlet port 410 to a support station 408 of the system housing 402. At 604, the handheld device 200 is suspended with a support station (such as the support station 108 shown in FIGS. 1 and 2 or other examples provided herein) in a hands free manner, for instance while the user disinfects or washes their hands. Optionally, at the support station (e.g., 408 of FIGS. 5A, B) the handheld device 200 is sterilized. In another example, the sterilization feature 316 of FIGS. 3A, B is a support station and suspends and sterilizes the handheld device 200.

At 606, the method 600 includes enclosing the handheld device 200 in a sanitary enclosure 114 of a plurality of sanitary enclosures 106 housed within a sanitary enclosure magazine 104. In one example, the sanitary enclosure magazine 104 includes a spool 107, shelf or other feature configured to receive the plurality of sanitary enclosures in a rolled or stacked configuration. In another example, the sanitary enclosure magazine 404 (as described and shown in FIG. 5B) includes an automated mechanism configured to dispense the sanitary enclosures. In still another example, enclosing the handheld device 200 includes sealing the device within the sanitary enclosure, for instance with an adhesive strip 118, a heat sealer 512, shrink wrapper or the like.

At 608, at least the hands of the user of the handheld device are disinfected. For instance, the user disinfects or washes their hands at a sink with soap, uses a sanitizing solution or the like. At 610, the handheld device enclosed within the sanitary enclosure is retrieved after disinfecting at least the hands of the user. After retrieval of the enclosed handheld device 200 the device may be used in a sanitary manner (e.g., for calls, interaction with applications, text messaging or mailing) in clean environments or when interacting with food items, patients or the like.

Several options for the method 600 follow. In one example, suspending the handheld device 200 with the support station 108 includes hanging the handheld device in the sanitary enclosure from an enclosure hanger 112. In another example, suspending the handheld device 200 with the support station 408 includes suspending the handheld device along a conveying surface 412 of the system housing 402. In still another example, the method 600 includes delivering the handheld device 200 from the support station 408 toward the sanitary enclosure 502 through a delivery port 416. Optionally, delivering the handheld device 200 includes moving a conveying stop 414 out of engagement with the handheld device 200.

In another example, the method 600 includes sterilizing the handheld device 200 with one or more sterilizing features. The sterilizing features include, but are not limited to LTV lamps, sanitizing liquid or aerosol dispensers, wiping or brush sanitizing applicators or the like. Optionally, sterilizing the handheld device 200 is conducted before enclosing the handheld device in the sanitary enclosure. In another option, sterilizing the handheld device 200 is concluded at the time the device 200 is enclosed in the sanitary enclosure, for instance with the sanitizer source 510 used in combination with the pressurized fluid source 508.

In still another example, enclosing the handheld device 200 in the sanitary enclosure includes positioning the handheld device within the sanitary enclosure through an enclosure opening 116 (e.g., an enclosure mouth). Optionally, enclosing the handheld device 200 includes closing the sanitary enclosure 114 (or 502) with the handheld device 200 therein with an enclosure sealer including, but not limited to, an adhesive strip 118, heat sealer 512, heating element configured to shrink wrap the enclosure material or the like.

### Examples

Example 1 can include subject matter such as an apparatus, such as can include a handheld device sanitary enclosure system comprising: a system housing including a sanitary enclosure magazine configured to hold a plurality of sanitary enclosures; a support station coupled with the system housing, the support station configured to hold and suspend a handheld device at the support station in a hands free manner; and an enclosure sealer configured to close a sanitary enclosure of the plurality of sanitary enclosures with a handheld device within the sanitary enclosure.

Example 2 can include, or can optionally be combined with the subject matter of Example 1, to optionally include wherein the support station includes an enclosure hanger of the system housing, and the enclosure hanger is coupled with the sanitary enclosure to hold and suspend a handheld device within the sanitary enclosure.

Example 3 can include, or can optionally be combined with the subject matter of one or any combination of Examples 1 or 2 to optionally include wherein the enclosure hanger includes a sanitary enclosure clamp, the sanitary enclosure clamp grasps the sanitary enclosure adjacent to an enclosure opening of the sanitary enclosure.

Example 4 can include, or can optionally be combined with the subject matter of one or any combination of Examples 1 through 3 to optionally include wherein the support station includes: a conveying surface of the system housing, and a conveying stop positioned along the conveying surface, wherein the conveying stop cooperates with the conveying surface to hold and suspend a handheld device positioned along the conveying surface.

Example 5 can include, or can optionally be combined with the subject matter of one or any combination of Examples 1-4 optionally to include wherein the conveying stop is movably positioned relative to the conveying surface to selectively arrest and allow movement of the handheld device.

Example 6 can include, or can optionally be combined with the subject matter of Examples 1-5 to optionally include a sterilization feature adjacent to the conveying surface, the sterilization feature configured to sterilize the handheld device.

Example 7 can include, or can optionally be combined with the subject matter of Examples 1-6 to optionally include a sterilization feature coupled with the system housing, the sterilization feature configured to sterilize the handheld device.

Example 8 can include, or can optionally be combined with the subject matter of Examples 1-7 to optionally include wherein the sterilization feature includes an ultraviolet light chamber having one or more ultraviolet light sources.

Example 9 can include, or can optionally be combined with the subject matter of Examples 1-8 to optionally include wherein the sterilization feature includes one or more of an ultraviolet source, a disinfectant applicator, or a disinfectant media.

Example 10 can include, or can optionally be combined with the subject matter of Examples 1-9 to optionally include wherein the plurality of sanitary enclosures includes a roll of interconnected sanitary bags, and the roll is rotatably coupled within the sanitary enclosure magazine.

Example 11 can include, or can optionally be combined with the subject matter of Examples 1-10 to optionally include wherein the plurality of sanitary enclosures includes a pliable elongate tube wrapped around an axis, the pliable elongate tube including a plurality of enclosure openings.

Example 12 can include, or can optionally be combined with the subject matter of Examples 1-11 to optionally include wherein the enclosure sealer includes a heat sealer.

Example 13 can include, or can optionally be combined with the subject matter of Examples 1-12 to optionally include wherein the enclosure sealer includes an adhesive feature.

Example 14 can include, or can optionally be combined with the subject matter of Examples 1-13 to optionally include wherein the enclosure sealer includes a shrink wrap sealer.

Example 15 can include, or can optionally be combined with the subject matter of Examples 1-14 to optionally include a handheld device sanitary enclosure system comprising: a system housing, the system housing including: a sanitary enclosure magazine configured to hold a plurality of sanitary enclosures, an inlet port extending toward at least one sanitary enclosure of the plurality of sanitary enclosures, and a sterilization feature interposed between the inlet port and the at least one sanitary enclosure; a support station interposed between the inlet portion and the at least one sanitary enclosure, the support station configured to hold and suspend a handheld device at the support station in a hands free manner; and an enclosure sealer configured to close a sanitary enclosure of the plurality of sanitary enclosure with a handheld device within the sanitary enclosure.

Example 16 can include, or can optionally be combined with the subject matter of Examples 1-15 to optionally include wherein a handheld device is retained in a sanitizing configuration and an enclosing configuration, in the sanitizing configuration, the handheld device is received through the inlet port and sterilized by the sterilization feature, and in the enclosing configuration, the handheld device is positioned within the sanitary enclosure of the plurality of sanitary enclosures and the sanitary enclosure is closed with the enclosure sealer.

Example 17 can include, or can optionally be combined with the subject matter of Examples 1-16 to optionally include wherein the support station includes: a conveying surface in communication with the inlet port, and a conveying stop positioned along the conveying surface, and in the sanitizing configuration the conveying stop cooperates with the conveying surface to hold and suspend a handheld device positioned along the conveying surface.

Example 18 can include, or can optionally be combined with the subject matter of Examples 117 to optionally include wherein the conveying stop is movably positioned relative to the conveying surface to selectively arrest and allow movement of the handheld device toward the sanitary enclosure.

Example 19 can include, or can optionally be combined with the subject matter of Examples 1-18 to optionally include wherein the support station includes a delivery port interposed between the sterilization feature and the sanitary envelope, the delivery port configured to deliver the handheld device into the sanitary enclosure.

Example 20 can include, or can optionally be combined with the subject matter of Examples 1-19 to optionally include wherein each of the plurality of sanitary enclosures includes an enclosure mouth configured to receive the handheld device, and the enclosure mouth of the sanitary enclosure of the plurality of sanitary enclosures is positioned adjacent to the delivery port.

Example 21 can include, or can optionally be combined with the subject matter of Examples 1-20 to optionally include wherein the sterilization feature includes an ultraviolet light chamber having one or more ultraviolet light sources.

Example 22 can include, or can optionally be combined with the subject matter of Examples 1-21 to optionally include wherein the sterilization feature includes one or more of an ultraviolet source, a disinfectant applicator, or a disinfectant media.

Example 23 can include, or can optionally be combined with the subject matter of Examples 1-22 to optionally include wherein the enclosure sealer includes a heat sealer downstream from the support station.

Example 24 can include, or can optionally be combined with the subject matter of Examples 1-23 to optionally include an outlet port, and a handheld device delivered through the outlet port is enclosed within the sanitary enclosure.

Example 25 can include, or can optionally be combined with the subject matter of Examples 1-24 to optionally include wherein the enclosure sealer includes a shrink wrap sealer.

Example 26 can include, or can optionally be combined with the subject matter of Examples 1-25 to optionally include a method for using a handheld device sanitary enclosure system comprising: delivering a handheld device to a system housing of the handheld device sanitary enclosure system; suspending the handheld device with a support station of the handheld device sanitary enclosure system in a hands free manner; enclosing the handheld device in a sanitary enclosure of a plurality of sanitary enclosures housed within a sanitary enclosure magazine; disinfecting at least the hands of the user of the handheld device; and retrieving the handheld device enclosed within the sanitary enclosure after disinfecting at least the hands of the user.

Example 27 can include, or can optionally be combined with the subject matter of Examples 1-26 to optionally include wherein delivering the handheld device to a system housing includes delivering the handheld device through an inlet port to the support station.

Example 28 can include, or can optionally be combined with the subject matter of Examples 1-27 to optionally include sterilizing the handheld device at the support station.

Example 29 can include, or can optionally be combined with the subject matter of Examples 1-28 to optionally include wherein suspending the handheld device with the support station includes hanging the handheld device in the sanitary enclosure from an enclosure hanger.

Example 30 can include, or can optionally be combined with the subject matter of Examples 1-29 to optionally include wherein suspending the handheld device with the support station includes suspending the handheld device along a conveying surface of the system housing.

Example 31 can include, or can optionally be combined with the subject matter of Examples 1-30 to optionally include delivering the handheld device from the support station toward the sanitary enclosure through a delivery port.

Example 32 can include, or can optionally be combined with the subject matter of Examples 1-31 to optionally include wherein delivering the handheld device includes moving a conveying stop out of engagement with the handheld device.

Example 33 can include, or can optionally be combined with the subject matter of Examples 1-32 to optionally include sterilizing the handheld device.

Example 34 can include, or can optionally be combined with the subject matter of Examples 1-33 to optionally include wherein sterilizing the handheld device is before enclosing the handheld device in the sanitary enclosure.

Example 35 can include, or can optionally be combined with the subject matter of Examples 1-34 to optionally include wherein enclosing the handheld device in the sanitary enclosure includes positioning the handheld device within the sanitary enclosure through an enclosure mouth.

Example 36 can include, or can optionally be combined with the subject matter of Examples 1-35 to optionally include wherein enclosing the handheld device includes closing the sanitary enclosure with the handheld device therein with an enclosure sealer.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the invention can be practiced. These embodiments are also referred to herein as “examples,” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more,” in this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (pr one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A handheld device sanitary enclosure system comprising:
a system housing including a sanitary enclosure magazine configured to hold a plurality of sanitary enclosures;
a support station coupled with the system housing, the support station configured to hold and suspend a handheld device at the support station in a hands free manner; and
an enclosure sealer configured to close a sanitary enclosure of the plurality of sanitary enclosures with a handheld device within the sanitary enclosure.

2. The handheld device sanitary enclosure system of claim 1, wherein the support station includes an enclosure hanger of the system housing, and the enclosure hanger is coupled with the sanitary enclosure to hold and suspend a handheld device within the sanitary enclosure.

3. The handheld device sanitary enclosure system of claim 1, wherein the enclosure hanger includes a sanitary enclosure clamp, the sanitary enclosure clamp grasps the sanitary enclosure adjacent to an enclosure opening of the sanitary enclosure.

4. The handheld device sanitary enclosure system of claim 1, wherein the support station includes:
a conveying surface of the system housing, and
a conveying stop positioned along the conveying surface, wherein the conveying stop cooperates with the conveying surface to hold and suspend a handheld device positioned along the conveying surface.

5. The handheld device sanitary enclosure system of claim 4, wherein the conveying stop is movably positioned relative to the conveying surface to selectively arrest and allow movement of the handheld device.

6. The handheld device sanitary enclosure system of claim 4 comprising a sterilization feature adjacent to the conveying surface, the sterilization feature configured to sterilize the handheld device.

7. The handheld device sanitary enclosure system of claim 1 comprising sterilization feature coupled with the system housing, the sterilization feature configured to sterilize the handheld device.

8. The handheld device sanitary enclosure system of claim 7, wherein the sterilization feature includes an ultraviolet light chamber having one or more ultraviolet light sources.

9. The handheld device sanitary enclosure system of claim 7, wherein the sterilization feature includes one or more of an ultraviolet source, a disinfectant applicator, or a disinfectant media.

10. The handheld device sanitary enclosure system of claim 1, wherein the plurality of sanitary enclosures includes a roll of interconnected sanitary bags, and the roll is rotatably coupled within the sanitary enclosure magazine.

11. The handheld device sanitary enclosure system of claim 1, wherein the plurality of sanitary enclosures includes a pliable elongate tube wrapped around an axis, the pliable elongate tube including a plurality of enclosure openings.

12. The handheld device sanitary enclosure system of claim 1, wherein the enclosure sealer includes a heat sealer.

13. The handheld device sanitary enclosure system of claim 1, wherein the enclosure sealer includes an adhesive feature.

14. The handheld device sanitary enclosure system of claim 1, wherein the enclosure sealer includes a shrink wrap sealer.

15. A handheld device sanitary enclosure system comprising:
a system housing, the system housing including:
a sanitary enclosure magazine configured to hold a plurality of sanitary enclosures,
an inlet port extending toward at least one sanitary enclosure of the plurality of sanitary enclosures, and
a sterilization feature interposed between the inlet port and the at least one sanitary enclosure;
a support station interposed between the inlet portion and the at least one sanitary enclosure, the support station configured to hold and suspend a handheld device at the support station in a hands free manner; and
an enclosure sealer configured to close a sanitary enclosure of the plurality of sanitary enclosure with a handheld device within the sanitary enclosure.

16. The handheld device sanitary enclosure system of claim 15, wherein a handheld device is retained in a sanitizing configuration and an enclosing configuration,
in the sanitizing configuration, the handheld device is received through the inlet port and sterilized by the sterilization feature, and
in the enclosing configuration, the handheld device is positioned within the sanitary enclosure of the plurality of sanitary enclosures and the sanitary enclosure is closed with the enclosure sealer.

17. The handheld device sanitary enclosure system of claim 16, wherein the support station includes:
a conveying surface in communication with the inlet port, and
a conveying stop positioned along the conveying surface, and in the sanitizing configuration the conveying stop cooperates with the conveying surface to hold and suspend a handheld device positioned along the conveying surface.

18. The handheld device sanitary enclosure system of claim 17, wherein the conveying stop is movably positioned relative to the conveying surface to selectively arrest and allow movement of the handheld device toward the sanitary enclosure.

19. The handheld device sanitary enclosure system of claim 15, wherein the support station includes a delivery port interposed between the sterilization feature and the sanitary envelope, the delivery port configured to deliver the handheld device into the sanitary enclosure.

20. The handheld device sanitary enclosure system of claim 19, wherein each of the plurality of sanitary enclosures includes an enclosure mouth configured to receive the handheld device, and the enclosure mouth of the sanitary enclosure of the plurality of sanitary enclosures is positioned adjacent to the delivery port.

21. The handheld device sanitary enclosure system of claim 15, wherein the sterilization feature includes an ultraviolet light chamber having one or more ultraviolet light sources.

22. The handheld device sanitary enclosure system of claim 15, wherein the sterilization feature includes one or more of an ultraviolet source, a disinfectant applicator, or a disinfectant media.

23. The handheld device sanitary enclosure system of claim 15, wherein the enclosure sealer includes a heat sealer downstream from the support station.

24. The handheld device sanitary enclosure system of claim 15 comprising an outlet port, and a handheld device delivered through the outlet port is enclosed within the sanitary enclosure.

25. The handheld device sanitary enclosure system of claim 15, wherein the enclosure sealer includes a shrink wrap sealer.

26. A method for using a handheld device sanitary enclosure system comprising:
delivering a handheld device to a system housing of the handheld device sanitary enclosure system;
suspending the handheld device with a support station of the handheld device sanitary enclosure system in a hands free manner;
enclosing the handheld device in a sanitary enclosure of a plurality of sanitary enclosures housed within a sanitary enclosure magazine;
disinfecting at least the hands of the user of the handheld device; and
retrieving the handheld device enclosed within the sanitary enclosure after disinfecting at least the hands of the user.

27. The method of claim 26, wherein delivering the handheld device to a system housing includes delivering the handheld device through an inlet port to the support station.

28. The method of claim 27 comprising sterilizing the handheld device at the support station.

29. The method of claim 26, wherein suspending the handheld device with the support station includes hanging the handheld device in the sanitary enclosure from an enclosure hanger.

30. The method of claim 26, wherein suspending the handheld device with the support station includes suspending the handheld device along a conveying surface of the system housing.

31. The method of claim 26 comprising delivering the handheld device from the support station toward the sanitary enclosure through a delivery port.

32. The method of claim 31, wherein delivering the handheld device includes moving a conveying stop out of engagement with the handheld device.

33. The method of claim 26 comprising sterilizing the handheld device.

34. The method of claim 33, wherein sterilizing the handheld device is before enclosing the handheld device in the sanitary enclosure.

35. The method of claim 26, wherein enclosing the handheld device in the sanitary enclosure includes positioning the handheld device within the sanitary enclosure through an enclosure mouth.

36. The method of claim 26, wherein enclosing the handheld device includes closing the sanitary enclosure with the handheld device therein with an enclosure sealer.
